(19) United States
(12) Patent Application Publication
(10) Pub. No.: US 2004/0137547 A1
(43) Pub. Date: Jul. 15, 2004

(54) Method for formulating a glucose oxidase enzyme with a desired property or properties and a glucose oxidase enzyme with the desired property

(75) Inventors: Rajiv Shah, Rancho Palos Verdes, CA (US); Bahar Reghabi, Marina Del Rey, CA (US)

Correspondence Address: FOLEY & LARDNER, 2029 CENTURY PARK EAST, SUITE 3500, LOS ANGELES, CA, 90067

(73) Assignee: Medtronic MiniMed, Inc.

(21) Appl. No.: 10/715,143

(22) Filed: Nov. 17, 2003

Related U.S. Application Data
(62) Division of application No. 10/035,918, filed on Dec. 28, 2001.

(51) Int. Cl. C12Q 1/54; C07H 21/04; C12N 1/16; C12N 1/18; C12N 9/02

(52) U.S. Cl. 435/14; 435/69.1; 435/189; 435/254.3; 435/320.1; 536/23.2; 435/252.33

(57) ABSTRACT

A method for formulating a glucose oxidase enzyme with peroxide resistant properties and a glucose oxidase enzyme formulated by the method. The enzyme formulation method results in a glucose oxidase enzyme with improve resistance to peroxide, and therefore, with improved resistance to oxidative inactivation. The method employs directed evolution techniques to evolve glucose oxidase to achieve the desirable properties. A peroxide resistant glucose oxidase may improve the longevity of, for example, glucose biosensors in which a peroxide resistant glucose oxidase may be placed.

This application is a Division of U.S. application Ser. No. 10/035,918, filed Dec. 28, 2001 which is incorporated herein by reference in its entirety. This application also claims the benefit under 35 USC 119(e) of U.S. Provisional Application No. 60/335,585, filed Oct. 23, 2001, which is incorporated herein by reference in its entirety.

### BACKGROUND OF THE INVENTION

1. Field of the Invention

The present invention relates, generally, to a method employing directed evolution techniques for formulating a glucose oxidase enzyme possessing a certain desired property or properties, and, in particular embodiments, for formulating a glucose oxidase enzyme having peroxide-resistant characteristics for use, by way of example, in a sensing device.

2. Description of the Related Art

The combination of biosensors and microelectronics has resulted in the availability of portable diagnostic medical equipment and has improved the quality of life for countless people. Many people suffering from disease or disability who, in the past, were forced to make routine visits to a hospital or a doctor's office for diagnostic testing currently perform diagnostic testing on themselves in the comfort of their own homes using equipment with accuracy to rival laboratory equipment.

Nonetheless, challenges in the biosensing field have remained. For example, although many diabetics currently utilize diagnostic medical equipment in the comfort of their own homes, the vast majority of such devices still require diabetics to draw their own blood and to inject their own insulin. Drawing blood typically requires pricking a finger. For someone who is diagnosed with diabetes at an early age, the number of self-induced finger-pricks and insulin injections over the course of a lifetime could reach into the tens of thousands. Drawing blood and injecting insulin thousands of times is overtly invasive and inconvenient and it can be painful and emotionally debilitating.

Diagnostic requirements of those with disease or disability may be addressed by using a sensing apparatus that may be implanted into the body and that may remain in the body for an extended period of time. An example implantable sensing system is disclosed in pending U.S. patent application Ser. No. 60/318,060, which is incorporated herein by reference. An example of the type of implantable sensing system described in that application contains a sensing device that is inserted into a vein, an artery, or any other part of a human body where it could sense a desired parameter of the implant environment. An enzyme may be placed inside of the sensing device and employed for sensing. For example, if physiological parameter sensing is desired, one or more proteins may be used as the matrix. If the device is a glucose-sensing device, then a combination of glucose oxidase (GOx) and human serum albumin (HSA) may be utilized to form a sensor matrix protein.

In a glucose sensing biosensor, for example, the sensor matrix protein is disposed adjacent to or near a metal electrode or electrodes that may detect oxygen electrochemically. The glucose oxidase works in the glucose sensor by utilizing oxygen to convert glucose to gluconic acid. A proposed mechanism of this reaction is illustrated in FIG. 1. As illustrated in FIG. 1, glucose complexes with the oxidized form of glucose oxidase (I). This complex renders itself into gluconic acid and the reduced form of an inactive glucose oxidase (IIa and IIb). The exact mechanism of this transformation is unknown. Two proposed mechanisms are illustrated in FIG. 1. One mechanism involves the hydride transfer from flavine adenine dinucleotide coenzyme (FAD). The other mechanism involves the formation of the glucosidic link. Glucose reacts as a catalyst to produce the active form of the reduced glucose oxidase (IV). This active form then reacts with oxygen, and glucose oxidase is oxidized (V) as a result.

The oxidation of glucose oxidase also results in the formation of a hydroperoxy adduct which transforms into hydrogen peroxide. As a result of this transformation, oxidized glucose oxidase is inactivated (VI). The inactive form will eventually become active (VII) and the cycle is repeated upon the reaction of another glucose molecule. The exact mechanism of this process is unknown.

An obstacle to creating sensors that are long-lived and stable over time has been that glucose oxidase, when immobilized (e.g., for use in a sensor) undergoes oxidative inactivation by the aforementioned peroxide over time. Since the lifetime of glucose sensors primarily depends on the lifetime of glucose oxidase, the effects of the peroxide on the glucose oxidase can severely limit the lifetimes of glucose sensors.

It is believed that immobilized glucose oxidase undergoes oxidative inactivation by peroxide over time because the peroxide attacks amino acids involved in binding either substrate or FAD. For example, methionine 561 is an amino acid that is involved in binding FAD to glucose oxidase. Since methionine 561 can be easily oxidized by peroxide, it might be a prime peroxide target.

Moreover, glucose oxidase binds glucose and uses oxygen to produce gluconic acid and peroxide. Hydroperoxy adducts are some of the intermediates in this process. The presence of such adducts along with oxygen and peroxide can result in superoxide radicals which, in effect, may attack both glucose and FAD binding sites. For example, Histidines 516 and 559 are prime peroxide targets. Both of these amino acids are involved in binding glucose. Oxidation of such amino acids may result in deactivation of the glucose oxidase.

Accordingly, there is a need in the industry for a glucose oxidase enzyme that is resistant to peroxide. Such an enzyme could, for example, be suitable for use in glucose biosensors because the enzyme's peroxide resistant properties might enhance the enzyme's longevity, and in turn, enhance the sensor's stability over time.

### SUMMARY OF THE DISCLOSURE

Therefore, it as an advantage of embodiments of the present invention to provide a method for formulating a glucose oxidase enzyme with desired properties, such as peroxide-resistant properties.

It is a further advantage of embodiments of the present invention that, while evolution under non-stress circumstances takes years, evolution may be manipulated in embodiments of the invention for specific biological characteristics or enzymatic functions. In embodiments of the invention, this technique, known as directed evolution, may be employed to evolve, for example, glucose oxidase in order to formulate a glucose oxidase that possesses improved resistance to oxidative damage, or, improved resistance to peroxide, or some other desired property.

It is a further advantage of embodiments of the present invention to provide a method for formulating glucose oxidase with improved peroxide-resistant properties that may be used, for example, in glucose biosensors. A glucose oxidase exhibiting improved peroxide resistance formulated pursuant to the method provided in the current invention may improve the longevity of a biosensor in which it is employed as compared to a glucose oxidase not formulated pursuant to the method provided herein.

In one embodiment of the invention, a method comprises obtaining a glucose oxidase gene or genes and employing the gene or genes to create a library of mutant genes or a library of variants. Each of the library of mutants is inserted into a separate expression vector. Each expression vector is then inserted into a host organism where a colony of the host organism can grow, thereby replicating the mutated genes. The library of colonies is then screened for desirable properties. In one embodiment, the screening procedures comprises screening for active glucose oxidase, screening for peroxide resistant properties, and then screening for functionality. In one embodiment, if, after the screening procedure, none of the colonies are found to be satisfactory, then the glucose oxidase from one or more of these colonies may be mutated into a second generation library of mutants. The process may then proceed again with the second generation mutations. In other embodiments, this same process may be repeated many times on subsequent generations of mutated genes until a gene is formulated with suitable properties.

Another embodiment of the invention involves, for example, a library of organisms, all of which contain glucose oxidase. In one embodiment, this library of organisms is grown in separate colonies with a conventional growth medium. In this embodiment, the environment of each colony is subsequently altered. For example, the environment of each colony may be altered by introducing peroxide to it. A screening procedure may be employed after the environments of the colonies have been altered. The screening procedure may involve processes of determining which of the colonies contain active glucose oxidase. Those colonies that still contain active glucose oxidase after their environments have been altered may possess desirable peroxide resistant qualities. Glucose oxidase from those colonies still containing active glucose oxidase may be tested for functionality, for example, by immobilizing the glucose oxidase in a sensor. In other embodiments of the invention, following at least a portion of the screening procedure, the environments of the colonies may be altered another time if desired. For example, in one embodiment, altering the environments of the colonies by adding more peroxide may reduce the number of colonies that proceed to the functionality testing.

These and other objects, features, and advantages of embodiments of the invention will be apparent to those skilled in the art from the following detailed description of embodiments of the invention, when read with the drawing and appended claims.

### BRIEF DESCRIPTION OF THE DRAWINGS

FIG. 1 shows a flow diagram of a glucose oxidase reaction sequence.

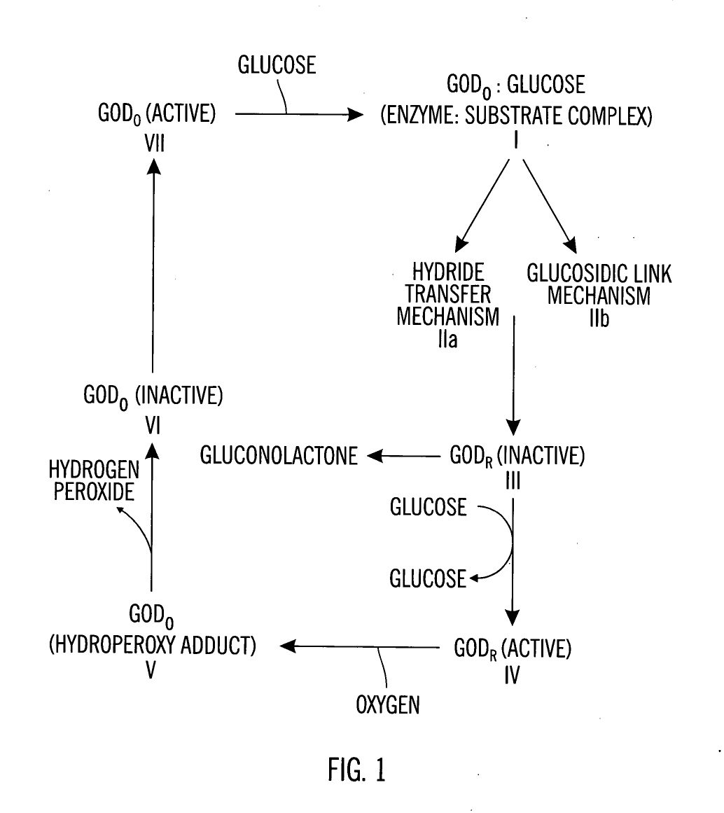

FIG. 2 shows a flowchart diagram of an embodiment of a method for formulating an enzyme With improved peroxide-resistant properties using directed evolution.

FIG. 3 shows a flowchart diagram of a screening procedure used in an embodiment of a method for formulating an enzyme with improved peroxide-resistant properties.

FIG. 4 shows a flowchart diagram of another embodiment of a method for formulating an enzyme with improved peroxide-resistant properties using directed evolution.

FIG. 5 shows a flow diagram of a directed evolution procedure according to one embodiment of the invention utilizing gene shuffling.

### DETAILED DESCRIPTION OF PREFERRED EMBODIMENTS

Embodiments of the invention are directed to processes for formulating a glucose oxidase enzyme with a particular desired property, such as, for example, an improved resistance to peroxide. Embodiments of the invention employ forced mutations that yield glucose oxidase enzymes that may or may not have an improved characteristic, such as an improved resistance to peroxide. Screening and/or testing procedures may be employed to assist in identifying mutated enzymes that might have desired qualities, such as peroxide resistant qualities. An enzyme derived from embodiments of the invention may be suitable for use, for example, in a biosensor. An enzyme derived from these embodiments may improve the performance and stability of a sensor.

Various biosensor configurations employ active enzymes as part of the sensor structure. Embodiments of the invention may be employed to produce active enzymes for various types of sensors. However, in one example embodiment, a process produces an enzyme for use in a sensor as described in co-pending U.S. Patent Application “Method For Formulating And Immobilizing A Matrix Protein And A Matrix Protein For Use In A Sensor,” filed Dec. 27, 2001, (attorney docket number 047711-0288).

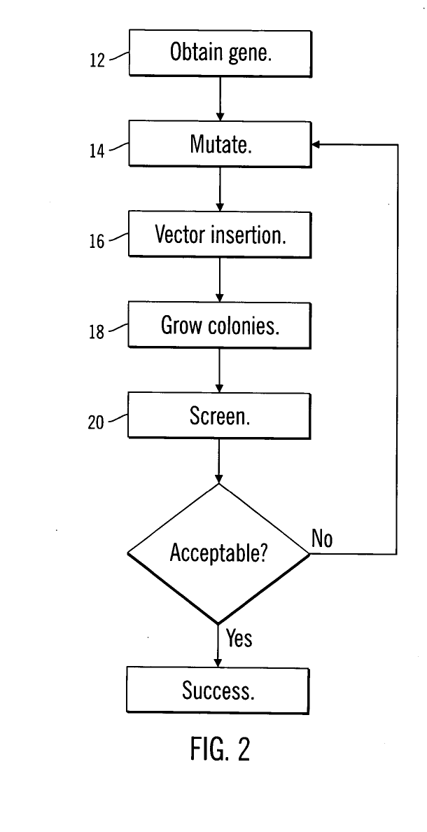

FIG. 2 shows a flowchart diagram of a process for utilizing a directed evolution procedure to formulate an enzyme having an improved resistance to peroxide, according to an embodiment of the invention.

Initially, the embodiment illustrated in FIG. 2 involves selecting or obtaining several glucose oxidase genes. The glucose oxidase genes can be taken from, for example, a yeast or a bacteria. In an example embodiment, the glucose oxidase genes are taken from Aspergillus Niger (“A. Niger”). However, in other embodiments, the genes could be derived from any member of a group including, but not limited to, A. Niger, Penecillium funiculosum, Saccharomyces cerevisiae, escherichia coli (E. Coli), and the like. Those skilled in the art will appreciate that the glucose oxidase genes could also be derived from other similar yeasts or bacteria.

Next in the example embodiment illustrated in FIG. 2, a library of mutant genes or variants may be created. In this context, a mutation refers to a random change in a gene or chromosome resulting in a new trait or characteristic that can be inherited. The process of creating a library of mutants represents a change in the enzyme. Mutation can be a source of beneficial genetic variation, or it can be neutral or harmful in effect. In these embodiments, the library of mutants may be created without necessarily knowing in advance whether any of the mutants will have the desired characteristics. The library of mutants or variants may be created in any of a number of ways. For example, the library of mutants could be created by procedures such as, but not limited to, Error-Prone Polymerase Chain Reaction (“Error-Prone PCR”), gene shuffling, and other like procedures.

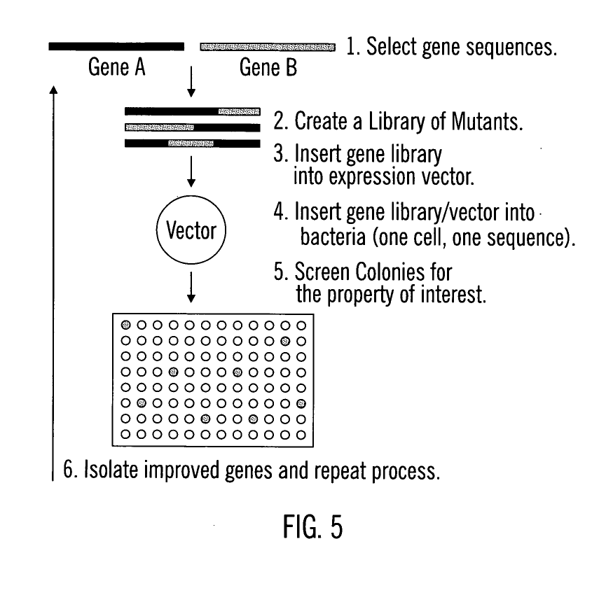

In one embodiment, Error-Prone PCR may be employed to create the library of mutant genes. Error-Prone PCR, as compared to PCR, has a relatively high rate of mutation. In other embodiments, the library of mutants may be created by a gene shuffling process. In the case of gene shuffling, a library of variants is created by recombining two or more parent genes. The recombined gene sequences may or may not yield functional enzymes. However, the functionality of the enzymes will be tested during the screening procedure. More importantly, the gene-shuffled library of variants will yield a suitable genetic diversity. FIG. 5 shows a flow diagram of a directed evolution procedure employing a gene-shuffling process for creating a library of mutants.

After at least a portion of the library of mutants has been created or assembled, the example embodiment in FIG. 2 involves inserting each of the mutated genes of the library of mutants into separate expression vectors. Generally, a gene may not be transferred directly from its original or source organism to a host organism. One way, however, to introduce a mutated gene into a host organism is to first introduce a gene into a vector. A vector is able to carry the gene into a host organism. Accordingly, at this point in the process of an example embodiment, each of the mutated genes may be inserted into an expression vector.

In the example embodiment of FIG. 2, each of the library of mutated genes which have been inserted into separate expression vectors are inserted into separate host organisms. The host organisms may be, for example, rapidly reproducing microorganisms which might be able to duplicate the recombined or mutated gene in large quantities. Some examples of suitable host organisms include E. Coli, A. Niger, and the like. Those skilled in the art will understand that other suitable host organisms are also available.

In an example embodiment, E. Coli may be employed as the host bacteria. In the example embodiment, once each of the library of mutants (in expression vectors) have been introduced into host organisms or bacteria, then each of the host organisms or bacteria may be placed into separate cells of a plate or tray. Within these separate cells, colonies of each of the host organisms or bacteria may be grown using any conventional growth medium. While a plate or tray with separate cells is used in the example embodiment, any other suitable holder or receptacle in which the host organisms or bacteria could grow would also work. For example, in another embodiment, each of the host organisms or bacteria could be placed in their own separate plates or trays.

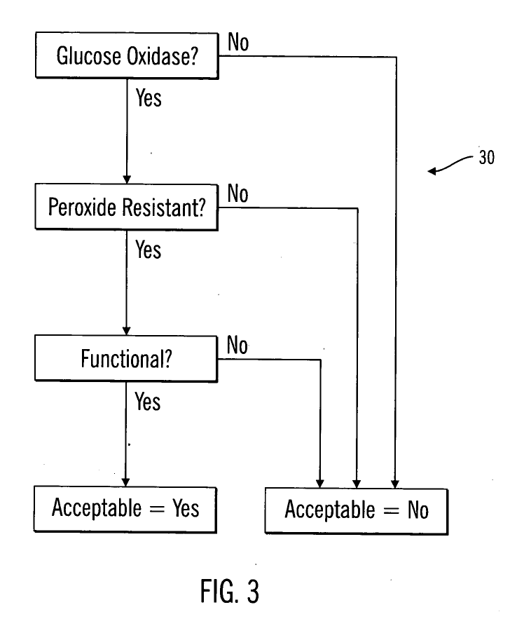

Once colonies of the host organisms or bacteria have grown, a screening procedure is employed in the example embodiment. In the example embodiment, the screening procedure is illustrated in FIG. 3. Initially, the screening procedure involves testing for glucose oxidase. A given colony may not necessarily yield active glucose oxidase following the gene mutation, the injection into the bacteria, and the growth process. Accordingly, the example embodiment includes determining whether the mutated genes that have been growing in the host organisms or bacteria yield active glucose oxidase. The test to determine whether a given colony contains active glucose oxidase may be conducted in any of a variety of ways. In one embodiment, the test for whether active glucose oxidase is present in a given colony comprises an assay which tests the production of peroxide. Peroxide is generated upon glucose oxidase reaction with glucose. In one embodiment, leuco-crystal-violet, a substrate that changes color in the presence of active peroxide, is employed. However, in other embodiments, other substances may also be used such as, but not limited to, aminoantipyrine, and the like.

In other embodiments, other methods can be used to test for the presence of active glucose oxidase. For example, the presence or absence of active glucose oxidase may be ascertainable by checking for fluorescence. The more fluorescent a given colony is, the more likely it is that it contains active glucose oxidase. Those skilled in the art will appreciate that further methods to test for the presence of glucose oxidase can be employed in other embodiments without deviating from the scope or spirit of the invention.

As illustrated in FIG. 3, if it is determined that a given colony does not contain active glucose oxidase, then the sample in that colony will not be acceptable because a goal of the process is to formulate a peroxide resistant glucose oxidase. Accordingly, in the example embodiment, for colonies in which active glucose oxidase is present, then the process proceeds to the next step in the screening procedure. For those colonies in which active glucose oxidase is not present, the process in concluded.

As illustrated in FIG. 2, the screening procedure in the example embodiment next involves determining whether the active glucose oxidase in the colonies that passed the first test in the screening procedure has peroxide-resistant properties. In the example embodiment, this portion of the screening procedure involves first incubating each remaining colony in peroxide. This may be done, for example, by placing a suitable amount of peroxide into the cells of the tray in which the colonies were grown. Other embodiments may introduce suitable amounts of peroxide to the various colonies other ways. For example, the peroxide may be introduced to the various colonies in separate trays or other receptacles.

After each of the remaining colonies has been incubated sufficiently with peroxide, the screening process then involves checking again for glucose oxidase activity. Specifically, after the peroxide incubation process, each colony may be tested for active glucose oxidase in similar ways as described above. Accordingly, after each of the remaining colonies has been incubated in peroxide, they may again be tested for glucose oxidase by, for example, using leuco-crystal-violet, a substrate which changes color in the presence of glucose oxidase. Other embodiments could use a different means for testing for active glucose oxidase without straying from the scope or spirit of the invention. Similarly, in other embodiments, the colonies could be incubated in peroxide and then tested for glucose oxidase activity one colony at a time or more than one colony at a time. In other words, it is not important to the invention that all colonies first be incubated in peroxide before any of the them can be tested for glucose oxidase.

In the example embodiment, if any of the remaining colonies tested negative for active glucose oxidase after the peroxide incubation process, then they may be deemed not acceptable. The colonies that still have active glucose oxidase, after being incubated in peroxide, may exhibit a desirable peroxide-resistive characteristic. As illustrated in FIG. 2, for the colonies that may exhibit the desirable peroxide-resistive characteristics, the screening procedure proceeds to the next step of testing functionality.

The screening procedure next involves determining whether a given glucose oxidase enzyme possesses the desired functionality. Thus, in embodiments in which the enzyme is being prepared for a biosensor, the procedure may involve testing whether a given glucose oxidase enzyme will work in a sensing device. In the example embodiment, this part of the screening procedure generally requires that the glucose oxidase be extracted from each of the remaining colonies. In the example embodiment, glucose oxidase may be extracted from the colonies using a purification column. Those skilled in the art will appreciate that there are other procedures available for extracting the glucose oxidase from the colonies for other embodiments of the invention.

In another embodiment, the process of assessing a given glucose oxidase enzyme's functionality may proceed as follows. First, cell lysis, or the removal of the protein from the source, may be achieved by a gentle grinding in a homogenizer. It can also be done by gentle disruption via sonication. Other embodiments might employ other means for removing the protein from the source. Next, the cell components may be subject to fractionation using centrifugation techniques and then differential solubility. The protein may subsequently be purified using standard chromatography methods. Next, the extracted protein may be characterized. This may be done by measuring the activity and concentration of the extract. Once the enzyme has been sufficiently isolated and sufficiently concentrated, then it may be immobilized and placed into a sensor. The sensor may then be introduced into an accelerated test environment to determine whether the particular enzyme is indeed functional or is suitable for use in a sensing device. If the results of the test with the enzyme in the sensor are satisfactory, then the testing can stop. This test may be repeated with every colony that exhibited peroxide resistant glucose oxidase after the incubation period. In other embodiments, this test could be done on a subset of those colonies depending on other factors or characteristics.

If a satisfactory glucose oxidase enzyme has not been identified after the screening procedure, then, in the embodiment illustrated in FIG. 2, the process may continue by creating another generation of mutated genes. In the example embodiment in FIG. 2, the entire cycle may be repeated as many times as desired.

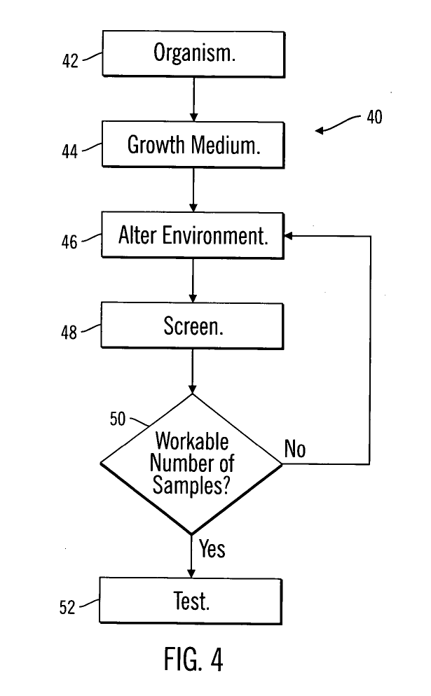

Another embodiment of the process of formulating an enzyme with peroxide-resistive properties is illustrated at FIG. 4. The example embodiment illustrated at FIG. 4 employs a forced mutation process. In this embodiment, instead of utilizing PCR or gene shuffling, mutations may be created by exposing organisms to harsh environments.

The embodiment in FIG. 4 first involves obtaining an organism, such as A. Niger, penecillium, E. Coli, or any other suitable organism. Since this embodiment will ultimately create a library of mutants as discussed above, the organism may be placed in multiple cells of a plate or tray. Other embodiments could employ other kinds of holders or receptacles in which to grow the organisms so long as the organisms are placed in separate colonies. Another embodiment of the invention may use only a single cell or colony. Next, this embodiment involves introducing a growth medium to each cell holding some of the organism. The growth medium may be any conventional growth medium such that the organisms may be sustained.

The embodiment in FIG. 4 next involves altering the environments of each of the separated organisms. In an embodiment in which the goal is to formulate a glucose oxidase enzyme with an enhanced peroxide resistance, the organisms' environments may be altered by adding a suitable amount of peroxide to each colony. In the example embodiment, the introduction of peroxide to the organisms' environments is done very gradually. In other embodiments, the introduction of peroxide to the organism's environment may be more abrupt.

The embodiment in FIG. 4 next involves a screening procedure. After peroxide has been added to the environments of the various colonies, the screening procedure may be employed to determine which of the colonies are still active. In this embodiment, the test discussed above may be employed for determining whether glucose oxidase in each of the colonies remains active. Other embodiments may employ other tests for determining whether a given colony contains active glucose oxidase.

At this point in the process, an assessment may be made as to whether the number of colonies with active glucose oxidase is such that the process may proceed to testing the glucose oxidase in sensing devices. Whether the number of remaining colonies is workable may depend on many factors and will vary for different embodiments of the invention. If a determination is made that there are too many remaining colonies to proceed to testing in sensing devices, then the environment may be made harsher by gradually adding more peroxide. In this embodiment, by repeating this cycle as many times as necessary, the environment may be continually and gradually made harsher until only a workable number of viable or active colonies remain.

In the example embodiment in FIG. 4, once the process yields a workable number of remaining colonies with active glucose oxidase, then the process may proceed to testing the glucose oxidase in sensing devices to assess functionality. The remaining colonies, which may possess the desirable peroxide resistant properties, may be tested for functionality as discussed above. In the example embodiment, this testing may be done by extracting glucose oxidase from the enzymes, incorporating the glucose oxidase in a sensor, and then effecting an accelerated test on the sensor to ascertain the functionality of the enzyme.

The embodiments disclosed herein are to be considered in all respects as illustrative and not restrictive of the invention. The scope of the invention is indicated by the appended claims, rather than the foregoing description. All changes that come within the meaning and range of equivalency of the claims are therefore intended to be embraced therein.

What is claimed is:

1. A method for formulating an enzyme comprising:
obtaining an organism with a glucose oxidase gene;
growing multiple colonies of the organism;
altering the environment of the colonies; and
screening the colonies to identify colonies with active glucose oxidase after altering the environment of the colonies.

2. A method for formulating an enzyme according to claim 1, wherein the organism is selected from a group consisting of Aspergillus Niger, Penecillium funiculosum, Saccharomyces cerevisiae, and Escherichia Coli.

3. A method for formulating an enzyme according to claim 1, wherein altering the environment of the colonies comprises introducing peroxide to the colonies.

4. A method for formulating an enzyme according to claim 1, wherein screening the colonies to identify colonies with active glucose oxidase comprises employing a substance that changes color in the presence of active glucose oxidase.

5. A method for formulating an enzyme according to claim 4, wherein the substance is leuco-crystal-violet.

6. A method for formulating an enzyme according to claim 5, wherein screening the colonies to identify colonies with active glucose oxidase comprises checking for fluorescence.

7. A method for formulating an enzyme according to claim 1, wherein the method further comprises testing the colonies with active glucose oxidase for functionality after screening the colonies to identify colonies with active glucose oxidase.

8. A method for formulating an enzyme according to claim 7, wherein the method further comprises continuing to alter the environments of the colonies until the colonies with active glucose oxidase are of a suitable number to proceed with testing the colonies with active glucose oxidase for functionality.

9. A method for formulating an enzyme according to claim 7, wherein testing the colonies with active glucose oxidase for functionality comprises employing glucose oxidase from the colonies in sensors.

10. A method for formulating an enzyme according to claim 7, wherein testing the colonies with active glucose oxidase for functionality comprises:
extracting glucose oxidase from the colonies;
immobilizing the glucose oxidase after extracting the glucose oxidase from the colonies;
placing the immobilized glucose oxidase in a sensor; and
testing the sensor.

11. A method for formulating an enzyme according to claim 10, wherein extracting glucose oxidase from the colonies comprises employing an ionic column to extract glucose oxidase from the colonies.

12. A method for formulating an enzyme according to claim 10, wherein extracting glucose oxidase from the colonies comprises:
removing the glucose oxidase from the colonies;
purifying the glucose oxidase; and
characterizing the glucose oxidase.

13. A method for formulating an enzyme according to claim 12, wherein removing the glucose oxidase from the colonies comprises grinding the colonies in a homogenizer into cell components.

14. A method for formulating an enzyme according to claim 13, wherein removing the glucose oxidase from the colonies further comprises fractionating the cell components employing centrifugation and differential solubility after grinding the colonies in a homogenizer.

15. A method for formulating an enzyme according to claim 12, wherein removing the glucose oxidase from the colonies comprises disrupting the colonies into cell components via sonication.

16. A method for formulating an enzyme according to claim 15, wherein removing the glucose oxidase from the colonies further comprises fractionating the cell components employing centrifugation and differential solubility after disrupting the colonies via sonication.

17. A method for formulating an enzyme according to claim 12, wherein purifying the glucose oxidase comprises purifying the glucose oxidase by employing chromatography methods.

18. An enzyme formulated according to the method of claim 1.
